(19) United States
(12) Patent Application Publication
(10) Pub. No.: US 2018/0228778 A1
(43) Pub. Date: Aug. 16, 2018

(54) COMPOSITIONS COMPRISING S1P RECEPTOR MODULATORS

(71) Applicant: Akaal Pharma Pty Ltd, Bundoora, Victoria (AU)

(72) Inventors: Gurmit S. Gill, Craigieburn, Victoria (AU); Damian W. Grobelny, Watsonia North, Victoria (AU)

(21) Appl. No.: 15/751,709

(22) PCT Filed: Aug. 11, 2016
(86) PCT No.: PCT/AU2016/050732

§ 371 Date: Feb. 9, 2018

(30) Foreign Application Priority Data
Aug. 11, 2015 (AU) 2015903210

(51) Int. Cl. A61K 31/4245; A61K 31/4439; A61K 31/4406; A61K 31/573; A61K 31/197; A61K 31/485; A61K 9/00; A61K 9/70; A61K 9/06; A61K 45/06; A61K 47/32

(52) U.S. Cl. CPC A61K 31/4245; A61K 47/32; A61K 31/4406; A61K 31/573; A61K 31/197; A61K 31/485; A61K 9/0014; A61K 9/0019; A61K 9/0046; A61K 9/0048; A61K 9/0053; A61K 9/7038; A61K 9/06; A61K 45/06; A61K 31/4439

(57) ABSTRACT

Compositions comprising S1P receptor modulators and at least one compound selected from one or more of the group consisting of steroids, opioids and non-steroidal anti-inflammatory drugs are provided. The compositions find use in the treatment of disease, particularly inflammation and immune mediated disorders.

### FIELD

This disclosure relates to compositions comprising S1P receptor modulators and to methods of treatment of disease, particularly inflammation and immune mediated disorders, using the compositions.

### BACKGROUND

Inflammation

Inflammation is an immune response to injury and infection. Symptoms include redness, heat, swelling and pain. The control of inflammation is important in regeneration and wound healing, however uncontrolled inflammation may give rise to a prolonged and damaging response resulting in chronic disease. Inflammation may be local or organ specific or it may spread over the body giving rise to systemic disease.

An inflammatory site has overexpressed pro-inflammatory cytokines and factors such as interleukins (IL1, IL6, IL17), tumour necrosis factor (TNFα), inducible-nitroxide-synthase (iNOS), cyclooxygenase-2 (COX-2), myeloperoxidase (MPO) and vascular endothelial growth factor (VEGF). These cytokines and factors are involved in the recruitment of immune cells, altered immune response, endothelial barrier disruption, altered differentiation and aberrant proliferation patterns within the inflammation. The blood vessels become abnormal, dilated, leaky, tortuous and there is fluid accumulation and edema. The remote parts are devoid of blood supply and develop hypoxia with possible onset of cancer. There is an altered pattern of cell survival and degeneration occurs.

An increasing body of scientific data reveals that inflammation is involved in nearly all ailments. Multiple diseases of inflammatory origin are common in various body organ systems such as cardiovascular (i.e. atherosclerosis, ischemic diseases, venous disease) nervous system (i.e. multiple sclerosis, epilepsy, ALS, neuropathy) and immune mediated diseases (i.e. rheumatoid arthritis, asthma, psoriasis, atopic dermatitis, acne, vitiligo). The inflammations have an underlying role in ischemic injury, atherosclerotic lesions (Galkina E. et al, Annu Rev Immunol, 2009, 27, 165) and cancer tumours (Landskron G. et al, J Immunol Res, 2014, Article ID 149185, 19 pages http://dx.doi.org/10.1155/2014/149185).

### S1P Receptor Axis and Inflammation

S1P receptors are a family of G-protein-coupled receptors with a wide range of expression over major organ systems such as immune, nervous and vascular systems. There are five receptors known as Sphingosine 1-phosphate receptors S1P1-5 with the common endogenous ligand S1P having a variety of downstream effects (Cooke et al, Annual Reports in Medicinal Chemistry, 2007, 42, pp 245-263 and references therein). The S1P receptors, especially the type 1 receptor S1P1, are involved in the immune response, endothelial barrier enhancement, (Wilkerson B A et al, J Biol Chem, 2012, Vol. 287, 44645) cellular protection (Rutherford C et al, Cell Death and Disease, 2013, 4, e927; doi:10.1038/cddis, 455), cell differentiation, cell mobilization/chemotaxis and others.

The downstream effect through the S1P receptor is known to involve the mTOR modulation and immune modulation (Liu G. et al, Nat Immunol, 2010, 11, 1047). S1P receptor involvement is well documented in the inhibition of the STAT3 (Garris C. S. et al, Nat Immunol, 2013, Vol 14, 1166) which is a known target involved in inflammations and cancer. The S1P receptors are well known to modulate pain (Welch S. P. et al, Biochem Pharmacol, 2012, 84, 1551). Further, S1P receptors are involved in stem cell chemotaxis (Kimura A. et al, Stem Cell, 2007, 25, 115) and regeneration (Leronimakis et al. Skeletal Muscle, 2013, 3, 20) and S1P1 axis is involved in neuroprotection (Asle R M et al, EXCLI Journal, 2013, 12, 449). S1P receptor modulation is involved in the expression of cytokines such as TNF_{α}, IL6, IL12, VEGF (Bolick D T et al, Arterioscler Thromb Vasc Biol, 2005, 25, 976; Sanchez T, et al, J Biol Chem 2003, 278 (47), 47281). S1P receptors have shown major involvement in critical illnesses such as acute lung injury, influenza and others. The endothelial cells make the inner layer of blood vessels express the S1P receptors and S1P1 agonists are well known to enhance the vascular barrier and prevent vascular leakage and enhance vasculature maturation (McVerry B. J. et al, J Cell Biochem, 2004, 92, 1075; Allende M. L. et al, Blood, 2003, 102, 3665; Paik J. et al, Genes Dev, 2004, 18, 2392; Garcia J. G. N. et al, J Clin Invest, 2001, 108(5), 689). The S1P receptor axis is involved in inflammations and cancer (Kunkel G. T. et al, Drug Discovery, 2013, 12, 688).

Diseases such as rheumatoid arthritis, joint pain, muscle inflammation, psoriasis, dermatitis, uveitis, and atherosclerosis are accompanied by inflammation, along with other co-pathologies such as pain, itching and degeneration. S1P receptors are known targets for multiple pathologies occurring in inflammatory indications.

Cancer of various origins has common pathologies such as inflammation, vascular abnormalities (leaky vessels, neo angiogenesis), hypoxia, aberrant differentiation, extravasation of cells from the primary place of cancer and metastasis. S1P receptor modulation may alleviate the multiple pathologies found in various cancers in a single treatment by alleviating inflammation, barrier enhancement, avoiding metastasis and cell differentiation. S1P receptor mediated cell clamping is reported to give a solid mass avoiding cell intravasation from the point of cancer (Feng H, Cancer Cell, 2010, 18(4), 353-366).

Vascular diseases have underlying causes of inflammatory response such as aberrant blood vessels, leaky and fluid extravasation and edema hyper vascularity. Neurodegeneration, inflammation and vascular leak, and hyper vascularity are common in macular degeneration, glaucoma, retinopathy. Lung inflammation is a central reason for various pulmonary problems such as asthma, chronic obstructive pulmonary disease (COPD), acute lung injury and influenza.

Myocardial infraction, spinal injuries and ischemic injuries are related to inflammation, cell death and compromised function of critical organs. S1P receptor modulation can alleviate the pathologies by halting inflammation, rescuing the cell death, (Schabbauer G. et al, Arterioscler Thromb Vasc Biol, 2004, 24, 1963; Wang J. et al., Biomaterials, 2015, 62, 76), improving flow of blood, attracting the stem cell to the site of injury, differentiation and regeneration (Leronimakis et al, Skeletal Muscle, 2013, 3, 20). The use of S1P receptor modulators can be extended to wound healing and regeneration of muscle, bone and other organs including transplant success (Lia L et al, Cornea, 2014, 33 (4), 398).

S1P receptor modulation is capable of addressing multiple pathological events (FIG. 1) common in various diseases of humans, animals and other species.

However, despite the abovementioned developments in S1P receptor modulation a need exists for improved S1P receptor modulators, for example those with S1P receptor subtype 1 activity, and particularly compositions containing S1P receptor modulators which are formulated to treat specific conditions.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

### SUMMARY

In one aspect there is provided a composition comprising at least one S1P receptor modulator and at least one compound selected from one or more of the group consisting of steroids, opioids and non-steroidal anti-inflammatory drugs.

The at least one S1P receptor modulator may be a compound of formula (I):

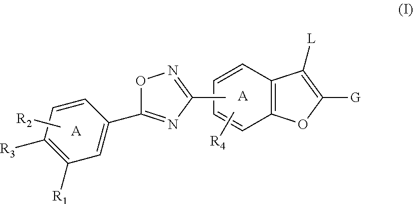

wherein R₁ is selected from hydrogen, deuterium, halogen, CN, CF₃, —COOH, amide, sulphonamide, aryloxy, nitro and an alkyl chain (C₁₋₅), said alkyl chain optionally containing one or more of deuterium, O, S, NR′ (R′═H, alkyl, cycloalkyl), halogen, a multiple bond, heterocycle, aryl, cycloalkyl (C₃₋₇) and carbocycle;

wherein R₂ is selected from hydrogen, deuterium, halogen, CN, CF₃, an alkyl chain (C₁₋₄) said alkyl chain optionally containing one or more of deuterium, O, S, NR′ (R′═H, alkyl, cycloalkyl), halogen, a multiple bond, heterocycle, aryl or cycloalkyl (C₃₋₇) and carbocycle;

wherein R₃ is selected from hydrogen, deuterium, halogen, an alkyl chain (C₁₋₇) said alkyl chain optionally containing one or more of deuterium, O, S, NR′ (R′═H, alkyl, cycloalkyl), halogen, a multiple bond, heterocycle, aryl or cycloalkyl (C₃₋₇) and carbocycle;

wherein R₄ is selected from hydrogen, deuterium, halogen, CN, CF₃, an alkyl chain (C₁₋₄) said alkyl chain optionally containing one or more of deuterium, O, S, NR′ (R′═H, alkyl, cycloalkyl), halogen, a multiple bond, heterocycle, aryl and cycloalkyl (C₃₋₇);

wherein A is optional and when present is selected to replace one or more ring carbon atoms by N;

wherein L is selected from hydrogen, deuterium, F, Cl, Br and alkyl (C₁₋₃);

wherein G is a group selected from one of the following:

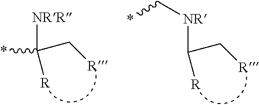

wherein R is selected from H, COOH, alkyl (C₁₋₄) and hydroxy-alkyl (C₁₋₄);
wherein R′ and R″ are independently selected from H and alkyl (C₁₋₄);
wherein R′″ is selected from OH, —OPO₃H₂ and physiologically acceptable salts;
wherein

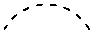

represents an optional bridging group;
the asterisks indicating the attachment of group G within formula (I).

The compound of formula (I) may have the formula (II):

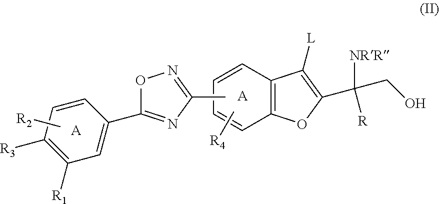

wherein R₁, R₂, R₃, R₄, A, L, R, R′ and R″ are as defined hereinbefore.

The compound of formula (I) or formula (II) may have:

R₁ selected from F, Cl, Br, CN, CF₃, NO₂, Me, OMe, OEt, OPr, O-iPr, O-isobutyl, O-isopentyl, O-cyclopentyl, O-allyl, O-benzyl and

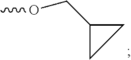

R₂ selected from H, deuterium, F, Cl, Br, CN, CF₃, NO₂, Me, OMe, OEt, OPr, O-iPr, O-isobutyl, O-isopentyl, O-cyclopentyl, O-allyl, O-benzyl and

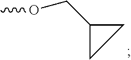

R₃ selected from H, deuterium, Pr, butyl, OMe, OEt, OPr, OiPr, O-isobutyl, O-isopentyl, O-butyl, O-pentyl, O-cyclopentyl, O-allyl, O-benzyl and

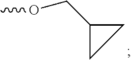

R₄ selected from H, deuterium, Me and Et;
R selected from H, Me or —CH₂OH;
R′ selected from H and Me;
R″ selected from H and Me;
L selected from H, deuterium, Me and Cl; and
A as defined hereinbefore.

The compound of formula (I) or formula (II) may have:

R₁ selected from F, Cl, Br, CN, CF₃, Me, NO₂, OMe, OEt, OPr, O-iPr, O-isobutyl, O-isopentyl, O-cyclopentyl, O-allyl, O-benzyl and

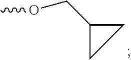

### R₂ is H;

R₃ selected from H, deuterium, Pr, butyl, OMe, OEt, OPr, OiPr, O-isobutyl, O-isopentyl, O-butyl, O-pentyl, O-cyclopentyl, O-allyl, O-benzyl and

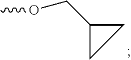

R₄ selected from H, deuterium, Me and Et;
R selected from H, Me or —CH₂OH;
R′ selected from H and Me;
R″ selected from H and Me;

### L is H; and

A is not present.

The compound of formula (I) may have the formula (III):

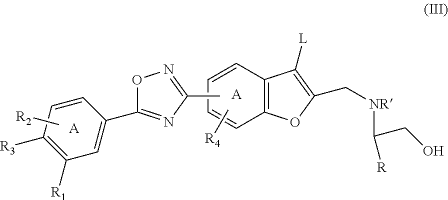

wherein R₁, R₂, R₃, R₄, A, L, R and R′ are as defined hereinbefore.

The compound of formula (I) may have the formula (III):

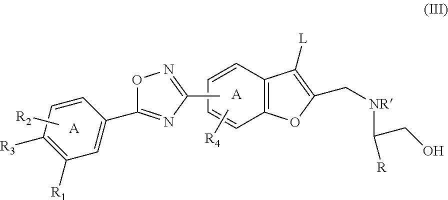

wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and

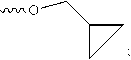

wherein R₂ is selected from H, deuterium, F, Cl, Br, CN, CF₃, Me, OMe, OEt, OPr, O-iPr, O-isobutyl, O-isopentyl, O-cyclopentyl, O-allyl, O-benzyl and

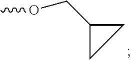

wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and

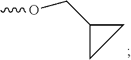

wherein R₄ is selected from H, deuterium, Me and Et;
wherein R is selected from H, Me or —CH₂OH;
wherein R′ is selected from H and Me;
wherein L is selected from H, deuterium, Me and Cl; and
wherein A is as defined hereinbefore.

The compound of formula (I) may have the formula (III)

wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and

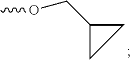

wherein R₂ is H;
wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and

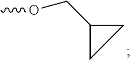

wherein R₄ is selected from H, deuterium, Me and Et;
wherein R is selected from H, Me or —CH₂OH;
wherein R′ is selected from H and Me;
wherein L is H; and
wherein A is not present.

The steroid may be a corticosteroid. The corticosteroid may be selected from the group consisting of aclometasone, amcinonide, beclomethasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumethasone, flunisolide, fluocinolone acetonide, fluocinonide, fluocortin, fluocortolone, fluorometholone, fluperolone, fluticasone, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone and ulobetasol, pharmaceutically acceptable salts, esters, solvates, hydrates and derivatives thereof, and mixtures thereof.

The corticosteroid may be betamethasone.

The opioid may be selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts, solvates, hydrates and derivatives thereof, and mixtures thereof.

The non-steroidal anti-inflammatory drug may be selected from the group consisting of aspirin, ibuprofen, naproxen, Diclofenac, Cox-2 inhibitors, etodolac, indomethacin, ketoprofen, piroxicam, folmetin, tenoxicam, mecoxicam, meloxicam, mefenamic acid, ibufenac, ketoprofen, methyl salicylate, pharmaceutically acceptable salts, solvates hydrates and derivatives thereof, and mixtures thereof.

The composition according to the present disclosure may comprise a compound formula (III):

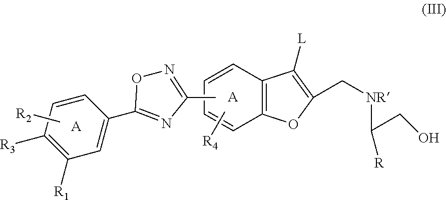

wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and

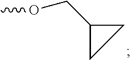

wherein R₂ is selected from H, deuterium, F, Cl, Br, CN, CF₃, Me, OMe, OEt, OPr, O-iPr, O-isobutyl, O-isopentyl, O-cyclopentyl, O-allyl, O-benzyl and

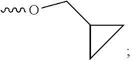

wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and

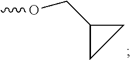

wherein R₄ is selected from H, deuterium, Me and Et;
wherein R is selected from H, Me or —CH₂OH;
wherein R′ is selected from H and Me;
wherein L is selected from H, deuterium, Me and Cl; and
wherein A is as defined hereinbefore; and a corticosteroid.

The corticosteroid may be betamethasone.

There is also provided a method of treating or preventing an inflammation mediated disorder, immune mediated disorder or pain by administering to a subject in need thereof an effective amount of a composition according to any one of the herein disclosed embodiments.

The inflammation mediated disorder or immune mediated disorder may be selected from the group consisting of psoriasis, eczema, vitiligo, alopecia, rheumatoid arthritis, osteoarthritis, gout, haemorrhoid/piles, lung injury, liver injury, kidney injury, asthma, chronic obstructive pulmonary disease (COPD), uveitis, retinopathy, nephropathy, macular degeneration, glaucoma, otitis, allergy, sepsis, influenza, rhinitis and pruritus.

There is also provided a method of treating or preventing pain by administering to a subject in need thereof an effective amount of a composition according to any one of the herein disclosed embodiments.

The pain may be selected from the group consisting of joint pain, arthritis, gout pain, back pain, muscle pain, muscle aches, neuropathy, neuralgia, myalgia, sports injury or wound pain.

In any of the herein disclosed methods the composition may be administered topically, orally, parenterally, intranasally, ocularly or rectally.

In any of the herein disclosed methods the composition may be in the form of a solid, a patch, a powder, a liquid, a semisolid, an ointment, a gel, a spray, an aerosol, a lotion, a tablet, a capsule, a liquid, a solution, a suspension, an emulsion or a syrup.

In any of the herein disclosed methods the composition may be a slow release formulation (depot preparation), administered by implantation or injection or device.

In any of the herein disclosed methods the composition may be administered in combination with other therapeutically active compounds, such as small molecules, biologicals, antivirals, antibacterials, anticancer drugs or anti-inflammatory agents.

In one embodiment there is provided a method of treating or preventing an inflammation mediated disorder, immune mediated disorder or pain by administering to a subject in need thereof an effective amount of a composition comprising a compound of formula (III):

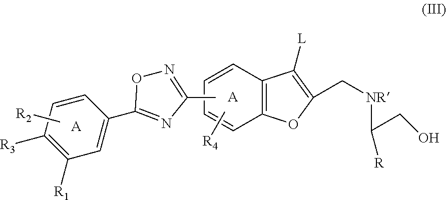

wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and

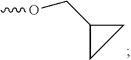

wherein R₂ is selected from H, deuterium, F, Cl, Br, CN, CF₃, Me, OMe, OEt, OPr, O-iPr, O-isobutyl, O-isopentyl, O-cyclopentyl, O-allyl, O-benzyl and

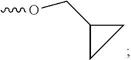

wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and

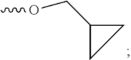

wherein R₄ is selected from H, deuterium, Me and Et;
wherein R is selected from H, Me or —CH₂OH;
wherein R′ is selected from H and Me;
wherein L is selected from H, deuterium, Me and Cl; and
wherein A is as defined hereinbefore; and a corticosteroid.

The corticosteroid may be betamethasone.

Representative examples of the compound of formula (I) include, but are not limited to, the following:

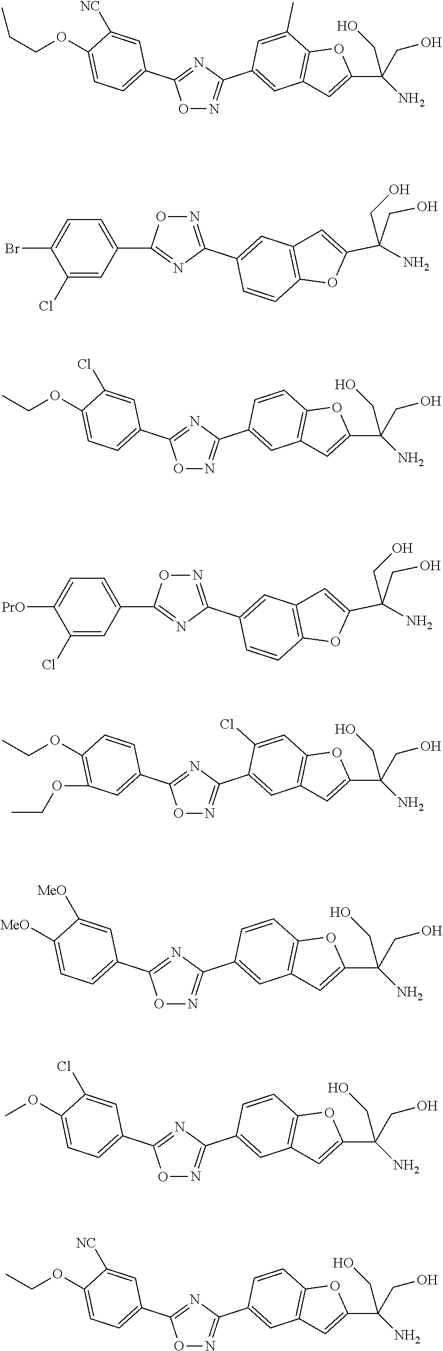

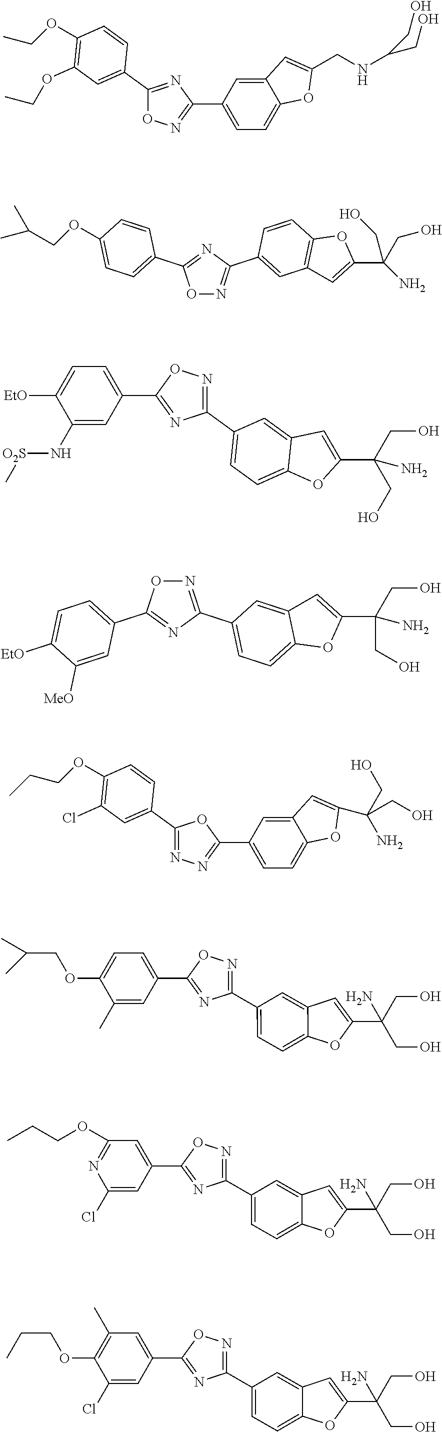

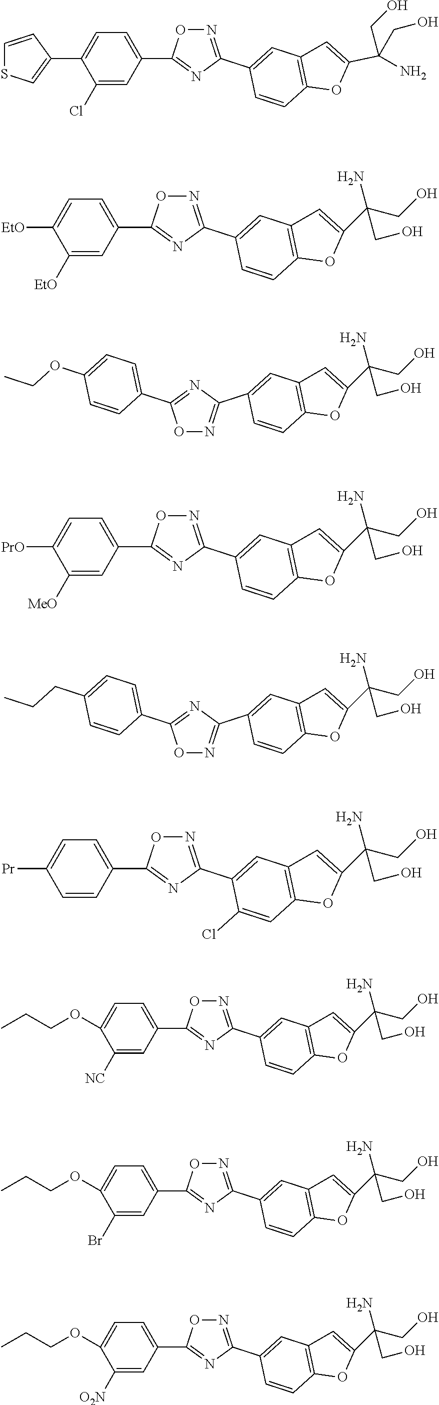

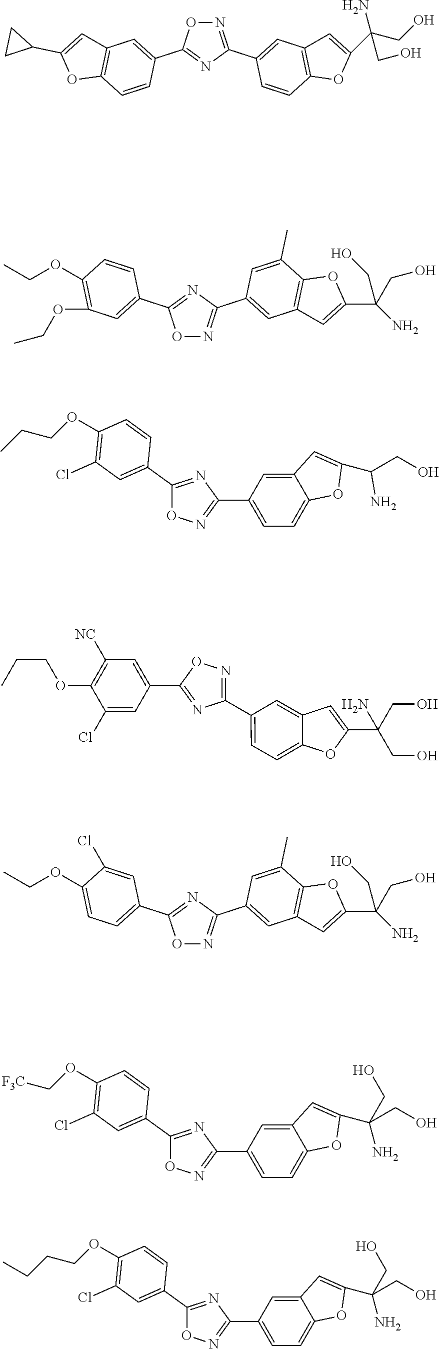

The S1P receptor modulator, for example the compounds of formula (I), may be in the form of salts. The salts of the S P receptor modulator, for example the compounds of formula (I), may be pharmaceutically acceptable. Suitable pharmaceutically acceptable salts will be apparent to those skilled in the art and include those described in J Pharm Sci, 1977, 66, 1-19, such as acid addition salts formed with inorganic acids for example hydrochloric, hydrobromic, sulfuric, nitric or phosphoric acid; and organic acids for example succinic, maleic, acetic, fumaric, citric, tartaric, benzoic, p-toluenesulfonic, methanesulfonic or naphthalenesulfonic acid. Certain S1P receptor modulators, for example the compounds of formula (I), may form acid addition salts with one or more equivalents of the acid. The present disclosure includes within its scope all possible stoichiometric and non-stoichiometric forms and free base forms.

The S1P receptor modulator, for example the compounds of formula (I), may be prepared in crystalline or non-crystalline form, and, if crystalline, may optionally be hydrated or solvated. This disclosure includes within its scope stoichiometric hydrates or solvates as well as compounds containing variable amounts of water and/or solvent and all salts, solvates, hydrates, complexes, polymorphs, prodrugs, radiolabeled derivatives, stereoisomers and optical isomers of the S1P receptor modulator, for example the compounds of formula (I).

The compositions according to the present disclosure may comprise a variety of delivery vehicles such as pharmaceutical excipients, including stabilizing agents, carriers or encapsulation formulations. The compositions may provide favourable synergistic effect between the one or more S1P receptor modulators, for example the compounds of formula (I), the at least one compound selected from one or more of the group consisting of steroids, opioids and non-steroidal anti-inflammatory drugs and the delivery vehicles. The synergistic effect may improve treatment and/or prevention and/or immunotherapy in comparison to the S1P receptor modulator, for example the compounds of formula (I), alone.

The compositions according to the present disclosure may be adapted for local or targeted use such as topical, ear, eye, nasal, oral, parenteral, rectal administration and, as such, may be in the form of compositions wherein the S1P receptor modulator, for example the compounds of formula (I), and at least one compound selected from one or more of the group consisting of steroids, opioids and non-steroidal anti-inflammatory drugs, are present as active, as active alone, or in various compositions in combination with other active and non-active ingredients/excipients such as and not limited to ointments, gels, hydrogel, solution, drops, topical patches, transdermal patches, topical liquid preparations, sprays, aerosols, tablets, capsules, oral liquid preparations, powders, granules, lozenges, controlled release particles including microparticles, liposomes, nano-emulsions, polymers, microsponges or fullerenes, injectable or infusible solutions or suspensions or suppositories and others.

Targeted dosing or application of the composition according to the present disclosure to an affected area may be advantageous over systemic exposure. This may result in a targeted and desired exposure of a S1P receptor modulator, for example the compounds of formula (I) and at least one compound selected from one or more of the group consisting of steroids, opioids and non-steroidal anti-inflammatory drugs, to the diseased part while avoiding the potential side effects that may occur by unnecessary exposure to healthy organs. For example a skin lesion of psoriasis or atopic dermatitis (eczema) may receive the required exposure to a S1P receptor modulator, for example the compounds of formula (I) and at least one compound selected from one or more of the group consisting of steroids, opioids and non-steroidal anti-inflammatory drugs, by direct administration to the lesion of a composition according to the present disclosure, while a systemic treatment may not achieve adequate therapeutic exposure of a S1P receptor modulator, for example the compounds of formula (I), and at least one compound selected from one or more of the group consisting of steroids, opioids and non-steroidal anti-inflammatory drugs, to the lesion and exposure to other non-targeted organs may cause adverse events.

The compositions according to the present disclosure may be slow releasing compositions and may allow the slow release of the S1P receptor modulator, for example the compounds of formula (I), and at least one compound selected from one or more of the group consisting of steroids, opioids and non-steroidal anti-inflammatory drugs. The release may be from an implant that may have a desirable therapeutic level of the S1P receptor modulator, for example the compounds of formula (I), in or at the periphery of an affected part, for example, inflammation, ischemic injury, cancer, tumor, atherosclerotic lesion and with associated low systemic concentration. The process may enhance the overall therapeutic window which otherwise may not be possible via systemic treatment. Accordingly, there is provided a method of treating inflammation, ischemic injury, cancer, tumor, or atherosclerotic lesion by local administration of an effective amount of a composition according to the present disclosure to a subject in need. The composition may be a slow release composition.

The compositions according to the present disclosure may be in various forms such as liquid, semisolid or solid, and various composition types such as solution, ointment, gel, paste, lotion, foam, controlled degrading polymers, patches, containing the S1P receptor modulator such as a compound of formula (I), and at least one compound selected from one or more of the group consisting of steroids, opioids and non-steroidal anti-inflammatory drugs. These various formulations may be used for targeted administration of a compound of formula (I) to treat the indications of body parts such as skin, eye, ear, nose, mouth, rectum, anus or lungs via direct applications; the gastrointestinal organs via a slow releasing formulation; the internal organs via implants, or injecting. Accordingly, there is provided a method of treating indications of body parts such as skin, eye, ear, nose, mouth, rectum, anus or lungs via direct applications; the gastrointestinal organs via a slow releasing formulation; the internal organs via implants, or injecting by administration of an effective amount of a composition according to the present disclosure to a subject in need.

Inflammatory indications are often associated with infections. Compositions of an S1P receptor modulator, for example the compounds of formula (I), and at least one compound selected from one or more of the group consisting of steroids, opioids and non-steroidal anti-inflammatory drugs, with an anti-infective or anti-pathogen agent such as an antibacterial, antiviral or antifungal are provided which may be used to treat such indications, for example, forms of eczema and acne. Accordingly, there is provided a method of treating indications such as eczema or acne by administration of an effective amount of a composition according to the present disclosure in combination with an anti-infective or anti-pathogen agent such as an antibacterial, antiviral or antifungal agent to a subject in need.

Steroid resistance can be restored by the addition of an S1P receptor modulator (Tsuji T. et al, Biol Pharm Bull, 2012, 35(8), 13149). S1P receptor modulation also has impact in the resistance development to Tamoxifen in cancer (Watson C et al, Am J Pathol, 2010, 177(5), 2205). Thus a composition comprising an S1P receptor modulator, for example the compounds of formula (I), and at least one compound selected from one or more of the group consisting of steroids, opioids and non-steroidal anti-inflammatory drugs, with other types of drugs such as, but not limited to, immune modulating agents, anticancer, antibacterial, antiviral, antifungal, pain modulators is provided.

There is provided a method of treating hypoxia, for example at the remote part of cancer, by local administration of an effective amount of a composition according to the present disclosure to a subject in need.

Transplant rejection is often accompanied by inflammation (Lutz et al, J Inflamm (Lond), 2010, 7, 27; Liang J et al, Cornea, 2014, 33 (4), 398). S1P receptor modulation is involved in an immune tolerance and vasculature correction and a local administration and optimal exposure is a promising approach for successful transplants with or without the other immune modulators. Accordingly, there is provided a method of treating transplant rejection by local administration of an effective amount of a composition according to the present disclosure to a subject in need.

S1P receptor modulation can mount an effective and appropriate response which spans from immune action against infection (Pinschewer D. D. et al, Neurology, 2011, 76 (Suppl 3): S15-S19) or cancer (Marcus A et al, Blood, 2011, 118(4), 975) to the immune tolerance (Liu G. et al, J Immunol, 2014, 192; Yoshida Y. et al, Biol Pharm Bull, 2011, 34(6), 933) and transplant success. Thus S1P receptor modulation has broad possible use where either the immune response is required against or in favour. This is surprising and may require an appropriate mode of dosing and compositions with the compound of formula (I) to obtain the desired effect.

There is also provided a composition comprising an S1P receptor modulator, for example the compounds of formula (I), and at least one compound selected from one or more of the group consisting of steroids, opioids and non-steroidal anti-inflammatory drugs, and highly specific proteins, peptides, or molecules, such as antibodies. The composition may be used for targeted delivery to a specific area or organ of the body.

The composition according to the present disclosure may be a topical composition. The topical composition may be in the form of a liquid formulation, such as and not limited to lotion and solution, semisolid formulations such as and not limited to ointment, gel, foam or cream, sprays and aerosols, or solid formulation such as and not limited to topical patches. The topical delivery systems may also include aerosol foams, liposomes, nano-emulsions, polymers, microsponges or fullerenes (Pharma Innovation, 2012, 1(9), 18-31). A topical composition may contain skin penetration enhancers. Examples of skin penetration enhancers include, but are not limited to short chain alcohols, such as ethanol and isopropanol; long chain alcohols such as decanol, hexanol, lauryl alcohol, myristyl alcohol, octanol, octyl dodecanol, oleyl alcohol; cyclic amides, such as azone; esters, such as ethyl acetate, octyl salicylate, padimate O, ethyl oleate, glyceryl monoleate, glyceryl monocaprate, glyceryl tricaprylate, isopropyl myristate, isopropyl palmitate, propylene glycol monolaurate, or propylene glycol monocaprylate; fatty acids such as lauric, linoleic, myristic, oleic, palmitic, stearic or isostearic acids; glycols such as dipropylene, propylene, 1,2-butylene or 1,3-butylene glycols; pyrrolidones, such as N-methyl-2-pyrrolidone, or 2-pyrrolidone; sulphoxides, such as decylmethyl sulphoxide or dimethyl sulphoxide; anionic surfactants such as sodium lauryl sulphate, cationic surfactants such as alkyl dimethylbenzyl ammonium halides, alkyl trimethyl ammonium halides, alkyl pyridinium halides, non-ionic surfactants, such as Brij 36T or Tween 80; monoterpenes, such as eugenol, d-limonene, menthol, menthone; sesquiterpenes, such as farnesol or neridol.

The composition of the present disclosure, comprising a S1P receptor modulator, for example the compounds of formula (I), and at least one compound selected from one or more of the group consisting of steroids, opioids and non-steroidal anti-inflammatory drugs, may comprise other ingredients, for example solubility enhancers or permeation enhancers such as and not limited to DMSO, polyvinyl pyrrolidones (PVPs), glycyryl laurates, lauryl lactate, aerosol, eudragit which may be dissolved in a solvent (for example ethanol, propanol or isopropanol). An adhesive may be added and mixed to give a homogenous mixture. The homogenous mixture may be cast onto a release layer, for example, silicone or fluoropolymer coated polyester film and dried.

The topical composition, tablets and capsules according to the present disclosure for oral administration may be in unit dose form, and may contain conventional excipients, such as binding agents (e.g. pregelatinised maize starch, polyvinylpyrrolidone, hydroxyethyl or hydroxypropyl methylcellulose); fillers (e.g. lactose, microcrystalline cellulose or calcium hydrogen phosphate); tableting lubricants (e.g. magnesium stearate, talc or silica); disintegrants (e.g. potato starch or sodium starch glycolate); and acceptable wetting agents (e.g. sodium lauryl sulphate). The tablets may be coated according to methods well known in normal pharmaceutical practice. The tablets may be slow releasing and release in specific organs, such as stomach or intestines, to deliver the S1P receptor modulator, for example the compounds of formula (I), and at least one compound selected from one or more of the group consisting of steroids, opioids and non-steroidal anti-inflammatory drugs.

The topical and oral liquid compositions of the present disclosure may be in the form of aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for topical and oral administration may be suitably formulated to give controlled release of the S1P receptor modulator, for example the compounds of formula (I), and at least one compound selected from one or more of the group consisting of steroids, opioids and non-steroidal anti-inflammatory drugs.

For parenteral administration, fluid unit dosage forms may be prepared utilizing a S1P receptor modulator, for example the compounds of formula (I) or pharmaceutically acceptable salts thereof, at least one compound selected from one or more of the group consisting of steroids, opioids and non-steroidal anti-inflammatory drugs, and a sterile vehicle. Formulations for injection may be presented in unit dosage form e.g. in ampoules or in multi-dose, utilizing a S1P receptor modulator, for example the compounds of formula (I), or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilized before filling into a suitable vial or ampoule and sealing. Advantageously, adjuvants such as a local anaesthetic, preservatives and buffering agents may be dissolved in the vehicle. To enhance the stability, the composition may be frozen after filling into the vial and the water removed under vacuum. Parenteral suspensions may be prepared in substantially the same manner, except that the compound is suspended in the vehicle instead of being dissolved, and sterilization cannot be accomplished by filtration. The compound may be sterilized by exposure to ethylene oxide before suspension in a sterile vehicle. Advantageously, a surfactant or wetting agent may be included in the composition to facilitate uniform distribution of the compound.

Lotions may be formulated with an aqueous or oily base and may also contain one or more emulsifying agents, stabilizing agents, dispersing agents, suspending agents, thickening agents, or coloring agents. Drops may be formulated with an aqueous or non-aqueous base also comprising one or more dispersing agents, stabilizing agents, solubilizing agents or suspending agents. They may also contain a preservative.

The S1P receptor modulator, for example the compounds of formula (I), or pharmaceutically acceptable salts thereof, and at least one compound selected from one or more of the group consisting of steroids, opioids and non-steroidal anti-inflammatory drugs, may also be formulated in rectal compositions such as suppositories or retention enemas, e.g. containing conventional suppository bases such as cocoa butter or other glycerides.

The S1P receptor modulator, for example the compounds of formula (I), or pharmaceutically acceptable salts thereof, and at least one compound selected from one or more of the group consisting of steroids, opioids and non-steroidal anti-inflammatory drugs, may also be formulated as depot preparations. Such long acting formulations may be administered by implantation (for example subcutaneously or intramuscularly, in-situ, at the periphery of inflammatory and/or injury site) or by intramuscular injection. Thus, for example, the S1P receptor modulator, for example the compounds of formula (I), and at least one compound selected from one or more of the group consisting of steroids, opioids and non-steroidal anti-inflammatory drugs, may be formulated with suitable polymeric or hydrophobic materials (for example as an emulsion in an acceptable oil) or ion exchange resins, or as sparingly soluble derivatives, for example, as a sparingly soluble salt.

For a formulation as controlled release particles the required amount of the S1P receptor modulator, for example the compounds of formula (I), and at least one compound selected from one or more of the group consisting of steroids, opioids and non-steroidal anti-inflammatory drugs, may be treated with a polymer, specifically biodegradable polymers which degrade in vivo, either enzymatically or non-enzymatically or both, to produce biocompatible, toxicologically safe by-products which are further eliminated by normal metabolic pathways. The choice of such biodegradable polymers includes for example poly lactic-coglycolic acid (PLGA) polyanhydrides, polycaprolactone (PCL), complex sugars (hyaluronan, hitosan) and inorganics (hydroxyapatite). For better delivery formulations that incorporate S1P modulator, including a compound of formula (I), and at least one compound selected from one or more of the group consisting of steroids, opioids and non-steroidal anti-inflammatory drugs, with various types of block copolymers of polyesters with poly ethylene glycol (PEG). PLGA/PEG block copolymers as diblock (PLGA-PEG) or triblock molecules with both ABA (PLGA-PEG-PLGA) and BAB (PEG-PLGA-PEG). These drug delivery devices may avoid the inconvenient surgical insertion of large implants and the injectable biodegradable and biocompatible PLGA particles (microspheres, microcapsules, nanocapsules, nanospheres) may be employed for controlled-release dosage forms. PLGA particles may contain an S1P modulator, including compounds of formula (I) alone or in combination with other therapeutically relevant drugs. The active ingredients may be released from polymeric devices either by diffusion through the polymer barrier, or by erosion of the polymer material, or by a combination of both diffusion and erosion mechanisms. PLGA may be easily processed and fabricated in various forms and sizes. PLGA should have biocompatibility, drug compatibility, suitable biodegradation kinetics and mechanical properties criteria.

For intranasal administration, the compounds of formula (I) or pharmaceutically acceptable salts thereof, and at least one compound selected from one or more of the group consisting of steroids, opioids and non-steroidal anti-inflammatory drugs, may be formulated as solutions for administration via a suitable metered or unitary dose device or alternatively as a powder mix with a suitable carrier for administration using a suitable delivery device. Thus compounds of formula (I) or pharmaceutically acceptable salts thereof, and at least one compound selected from one or more of the group consisting of steroids, opioids and non-steroidal anti-inflammatory drugs, may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof, and at least one compound selected from one or more of the group consisting of steroids, opioids and non-steroidal anti-inflammatory drugs, may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilized components.

The compositions according to the present disclosure may contain from 0.001% to 99% by weight, preferably from 0.001 to 60% by weight, more preferably from 0.01 to 25% by weight, of the S1P receptor modulator, for example the compound of formula (I), depending on the method of administration. The dose of the S1P receptor used in the treatment of the aforementioned disorders will vary in the usual way with the seriousness of the disorders, the weight of the sufferer, and other similar factors. However, as a general guide suitable unit doses may be 0.001 to 10000 mg, 1.0 to 500 mg or 1.0 to 200 mg of a compound of formula (I) and such unit doses may be administered more than once a day, for example two or three or more times a day.

The compositions according to the present disclosure may contain from 0.001% to 99% by weight, preferably from 0.01 to 25% by weight, of at least one compound selected from one or more of the group consisting of steroids, opioids and non-steroidal anti-inflammatory drugs depending on the method of administration. The dose of the at least one compound selected from one or more of the group consisting of steroids, opioids and non-steroidal anti-inflammatory drugs used in the treatment of the aforementioned disorders will vary in the usual way with the seriousness of the disorders, the weight of the sufferer, and other similar factors. However, as a general guide suitable unit doses may be 0.05 to 10000 mg, 1.0 to 500 mg or 1.0 to 200 mg of the at least one compound selected from one or more of the group consisting of steroids, opioids and non-steroidal anti-inflammatory drugs and such unit doses may be administered more than once a day, for example two or three or more times a day.

In one embodiment the S1P receptor modulator, for example the compound of formula (I), may be present in the composition according to the present disclosure in an amount of 0.001 to 25 wt. % and the steroid present in an amount of 0.005 to 2 wt. %, based on the total weight of the composition.

In one embodiment the S1P receptor modulator, for example the compound of formula (I), may be present in the composition according to the present disclosure in an amount of 1 to 3 wt. % and the steroid present in an amount of 0.01 to 0.05 wt. %, based on the total weight of the composition.

In one embodiment the S1P receptor modulator, for example the compound of formula (I), may be present in the composition according to the present disclosure in an amount of 1.0 or 1.5 or 2.0 or 2.5 or 3.0 or 3.5 or 4.0 or 4.5 or 5.0 wt. % and betamethasone present in an amount of 0.005 or 0.01 or 0.02 or 0.03 or 0.04 or 0.05 or 0.06 or 0.07 or 0.08 or 0.09 or 0.10 wt. %, based on the total weight of the composition.

In one embodiment the S1P receptor modulator, for example the compound of formula (I), may be present in the composition according to the present disclosure in an amount of 1.0 to 3.0 wt. % and betamethasone present in an amount of 0.01 to 0.05 wt. %, based on the total weight of the composition.

In one embodiment the S1P receptor modulator, for example the compound of formula (I), may be present in the composition according to the present disclosure in an amount of 0.001 to 25 wt. % and the opioid present in an amount of 0.01 to 20 wt. %, based on the total weight of the composition.

In one embodiment the S1P receptor modulator, for example the compound of formula (I), may be present in the composition according to the present disclosure in an amount of 0.001 to 25 wt. % and the non-steroidal anti-inflammatory drug (NSAID) present in an amount of 0.1 to 20 wt. %, based on the total weight of the composition.

In one embodiment the S1P receptor modulator, for example the compound of formula (I), may be present in the composition according to the present disclosure in an amount of 0.5 or 1.0 or 1.5 or 2.0 or 2.5 or 3.0 wt. % and ibuprofen or diclofenac present in an amount of 0.5 or 1.0 or 1.5 or 2.0 or 2.5 or 3.0 wt. %, based on the total weight of the composition.

In one embodiment the S1P receptor modulator, for example the compound of formula (I), may be present in the composition according to the present disclosure in an amount of 1 to 3 wt. % and ibuprofen or diclofenac present in an amount of 1 wt. % to 4 wt. %, based on the total weight of the composition.

In one embodiment the S1P receptor modulator, for example the compound of formula (I), may be present in the composition according to the present disclosure in an amount of 2 wt. % and ibuprofen or diclofenac present in an amount of 2 wt. %, based on the total weight of the composition.

In one embodiment the S1P receptor modulator, for example the compound of formula (I), may be present in the composition according to the present disclosure in an amount of 2 to 3 wt. % and capsaicin present in an amount of 0.01 to 2.5 wt. %, based on the total weight of the composition.

In one embodiment the S1P receptor modulator, for example the compound of formula (I), may be present in the composition according to the present disclosure in an amount of 2 or 3 or 4 wt. % and capsaicin present in an amount of 0.025 or 0.5 or 1 wt. %, based on the total weight of the composition.

In one embodiment the S1P receptor modulator, for example the compound of formula (I), may be present in the composition according to the present disclosure in an amount of 2 to 3 wt. % and lignocaine present in an amount of 0.5 to 10 wt. % based on the total weight of the composition.

In one embodiment the S1P receptor modulator, for example the compound of formula (I), may be present in the composition according to the present disclosure in an amount of 2 to 3 wt. % and lignocaine present in an amount of 1 to 5 wt. %, based on the total weight of the composition.

In any of the aforementioned embodiments disclosing an amount of a compound of formula (I) in the composition, the compound of formula (I) may be a compound of formula (III):

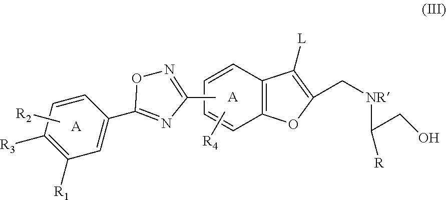

wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and

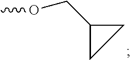

wherein R₂ is selected from H, deuterium, F, Cl, Br, CN, CF₃, Me, OMe, OEt, OPr, O-iPr, O-isobutyl, O-isopentyl, O-cyclopentyl, O-allyl, O-benzyl and

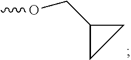

wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and

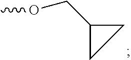

wherein R₄ is selected from H, deuterium, Me and Et;
wherein R is selected from H, Me or —CH₂OH;
wherein R′ is selected from H and Me;
wherein L is selected from H, deuterium, Me and Cl; and
wherein A is as defined hereinbefore

Compounds of formula (I) or pharmaceutically acceptable salts thereof may be used in combination preparations. For example, the compounds of formula (I) may be used in combination with other therapeutically active compounds, such as and not limited to cyclosporin A, methotrexate, steroids, corticosteroids, non-steroidal anti-inflammatory drugs, inflammatory cytokine inhibitors, kinase inhibitor (e.g., JAK Kinase), immunomodulators including biologicals, antivirals, including but not limited to aciclovir, 5-fluorouracil, galancyclovir, valancyclovir, vidaramine or zidovudine, and broad spectrum antiviral agents (Front Microbiol, 2015; 6: 517), antibiotics, including but not limited to amoxicillin, ceftaroline, colistin, dyptomycin, ertapenem, fosfomycin, penicillin, rapamycin or tigecyline; or antifungals, including but not limited to amphotericin, liposomal amphotericin B, fluconazole, flucytosine, micafungin, posaconasole and viriconazole.

Compounds of formula (I) or pharmaceutically acceptable salts thereof may be used in combination with other therapeutically active compounds such as opioids, corticosteroids, non-steroidal anti-inflammatory drugs, morphine, fentanyl, tramadol, methadone, oxycodone, indomethacin, diclofenac, ibuprofen, naproxen, celecoxib, amitriptyline, nortriptyline, desipramine, fluoxetine, paroxetine, duloxetine/venlafaxine, nabilone, gabapentin, cyclobenzaprine, baclofen, ketamine, ketoprofen, clonidine, verapamil.

The various compositions containing the compound/s of formula (I) or pharmaceutically acceptable salts thereof, and at least one compound selected from one or more of the group consisting of steroids, opioids and non-steroidal anti-inflammatory drugs, may be used in combinations with other excipients and/or active ingredients may be applied and spread over the affected part such as and not limited to skin lesions (psoriasis, eczema), wounds with the formulations, such as ointment, gel or lotion; and/or inhaled in to expose to the surface of affected part of lung, occurring in, but not limited to, a lung inflammation, chronic obstructive pulmonary disease (COPD) or asthma; and/or administered or implanted as a device with a control release in or at the periphery of affected part, such as, but not limited to, an atherosclerotic lesion, a cancer tumor, an ischemic injury or a transplant; and/or administered or implanted as device with a control release in or at the periphery of affected part requiring healing and/or regeneration, including but not limited to, bone regeneration, muscle regeneration, healing/regeneration at ischemic injury site, wounds.

The compositions according to the present disclosure may be in the form of eye drops. The drops may be used to treat eye disorders such as, but not limited to, eye inflammation, pain, retinopathy, macular degeneration, uveitis and glaucoma.

The compositions according to the present disclosure may be in the form of nasal drops. The drops may be used to treat indications such as but not limited to nasal inflammation, nasal congestion, rhinitis, nasal polyps, sinusitis, pain, migraine and headaches.

The compositions according to the present disclosure may be in the form of ear drops. The may be used to treat ear disorders such as but not limited to, ear inflammation, ear eczema, ear psoriasis, pain, chronic ulcer, wound, infection, and ear nerve regeneration.

The compositions according to the present disclosure may be in the form of inhalers. The inhalers may be used to treat lung disorders such as but not limited to lung inflammation, acute lung injury, asthma, COPD and pulmonary arterial hypertention.

The compositions according to the present disclosure may be used to treat skin disorders such as, but not limited to, psoriasis, eczema, dermatitis, cellulitis, actinic keratosis, acne, cutaneous T cell lymphoma, basal cell carcinoma, melanoma, vitiligo, wound, itch, pain, alopecia and dermal pain.

The compositions according to the present disclosure may be used to treat joint problems such as, but not limited to, joint inflammation, arthritis, rheumatoid arthritis, gout, and osteoarthritis.

The compositions according to the present disclosure may be used to treat injuries such as, but not limited to, ischemic injury, spinal cord injury, athlete injury, muscle injury and muscle cramp, muscle pain, muscle aches, or back pain. The compositions may be applied as a local application, local delivery, or implant in or at the periphery of injury/inflammation site.

The compositions according to the present disclosure may be used to treat vascular problems such as, but not limited to, hemorrhoids, blood vessel abnormalities and inflammations, vasculopathy, chronic wounds or leg ulcers.

The compositions according to the present disclosure may be used to treat pain such as, but not limited to, neuralgia, nociceptive pain, neuropathic pain, inflammatory pain, wound pain, tension headache, herpetic neuralgia, muscle pain, joint pain, back pain, wound pain, sports injury pain, and other pains.

The compositions according to the present disclosure may be used to treat gastrointestinal problems such as, but not limited to, gut inflammations, vessel abnormalities, wounds, ulcers, ulcerative colitis and Crohn's disease.

The compositions according to the present disclosure may be used for the treatment of atherosclerotic lesions. The compositions may be administered in or at the periphery of the lesion.

The compositions according to the present disclosure may be used for the treatment of cancer. The composition may be administered in or at the periphery of cancer, such as a cancer tumor.

The compositions according to the present disclosure may be used for bone regeneration, muscle regeneration, epithelial ulcer treatment, wound healing, therapeutic angiogenesis and gangrene treatment.

The compositions according to the present disclosure may be used in transplantation purposes, such as, but not limited to, cornea, kidney and liver transplants.

The compositions according to the present disclosure may be used for the treatment of inflammation and/or vascular abnormalities of the internal organs such as but not limited to kidney (nephropathy), prostate (prostatitis), urinary tract (inflammations), pancreases (pancreatitis), colon (colitis), liver (hepatic diseases, deep tissue (neuropathy, inflammations, degenerations), ulcers, wounds and ischemic injury. The formulation may be administered at, or at the periphery of, an affected area.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

### BRIEF DESCRIPTION OF THE DRAWINGS

FIG. 1 illustrates various S1P receptor modulator pathways.

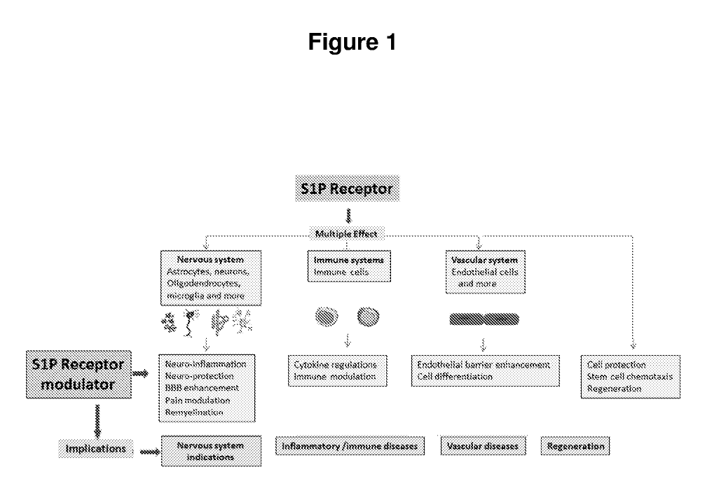

FIG. 2 illustrates the in vivo effect of oral AKP administration on dinitrofluorobenzene (DNFB)-induced delayed type hypersensitivity (DTH) in BALB/c mice.

FIG. 3 illustrates the topical efficacy of AKP (a compound of formula (I)), betamethasone or the combination in a mice model of Phorbol ester mediated irritant contact dermatitis.

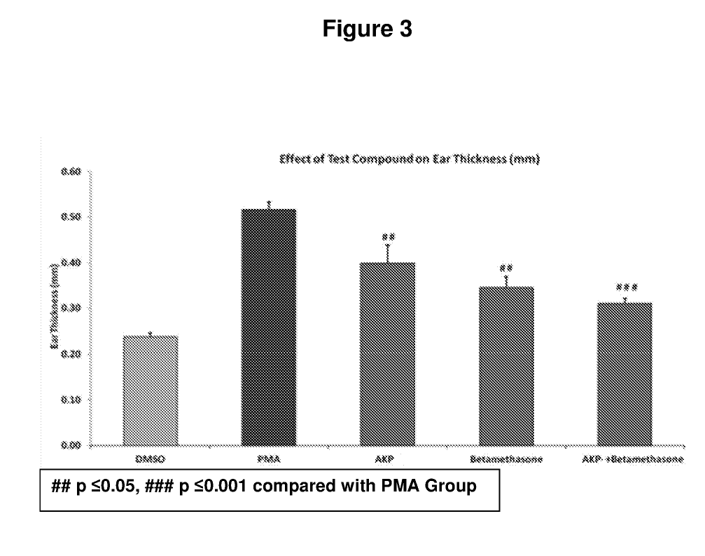

FIG. 4 illustrates the clinical effect of AKP (a compound of formula (I)) in a psoriasis study.

### DESCRIPTION OF EXEMPLARY EMBODIMENTS

Before the present compositions and/or methods are disclosed and described, it is to be understood that unless otherwise indicated this invention is not limited to specific compositions, components, designs, methods, or the like, as such may vary, unless otherwise specified. It is also to be understood that the terminology used herein is for the purpose of describing particular embodiments only and is not intended to be limiting.

### EXAMPLES

### Example 1

### Activity at Spingosine-1-Phosphate (S1P) Receptor

Compounds of formula (I) showed S1P receptor activity, especially the type 1 receptor agonistic activity. The S1P₁ assay system was GTPgama-S³⁵ binding in membranes from CHO K1 cells, expressing S1P₁ human receptor. The compounds were tested and generated a concentration-effect (dose response) curves at these receptors. The analysis provided efficacy (Eₘₐₓ) and potency (EC₅₀) of the compounds relative to S1P and demonstrated an EC₅₀ of <2 nM at the S1P₁ receptor. The compound of formula (I) has low tendency to degrade the S1P₁ receptor.

### Example 2

### Anti-Inflammatory Activity

A compound of formula (I) showed additional anti-inflammatory activities when tested on LPS challenged macrophages (RAW cells). The pro-inflammatory cytokines and factors are over expressed in inflammations, cancer and other indications. Lipopolysaccharide is well known to induce various pro-inflammatory factors in the monocytes (RAW cells) and the efficacy of compound on the LPS challenged cells was evaluated for the inhibition of tumor necrosis factor (TNFα), interleukins; IL1P, IL6, and factors such as inducible nitroxide synthase (iNOS), COX-2 and vascular endothelial growth factor (VEGF). The compound of formula (I) (AKP) markedly inhibited the multiple pro-inflammatory cytokines and factors including the permeability factor when used in-vitro in the range of 1 to 5 μM.

### Example 3

### Efficacy in Immune Mediated Skin Inflammatory Model

The formulation of representative compound of formula I have shown efficacy in a delayed-type hypersensitivity (DTH) reaction model. The DTH is an expression of cell-mediated immunity and plays a major role in the pathology and chronicity of many inflammatory disorders including inflammatory skin indications. One of the most characteristic DTH phenomena is contact hypersensitivity, which is characterized by swelling and increased tissue levels of cytokines. Contact hypersensitivity (CHS) is a T cell mediated immune reaction in response to cutaneous sensitization and challenge with reactive haptens those are capable of binding directly to soluble and cell associated proteins and recognized by T cells in the context of self-MHC products. The cells that recognize antigen-protein complex in the skin are the Langerhans cells (LCs). After topical application of an allergen, Langerhans cells (LCs), the major antigen-presenting cells (APCs) for the induction of immune responses in skin, show enhanced expression of surface MHC class II molecules, and start to emigrate from the skin to regional lymph nodes where specific lymphocyte activation is thought to occur. After a second contact with the haptens, T cells are first recruited into tissues and then activated by antigen-presenting cells to produce cytokines that mediate local inflammation. Myeloperoxidase (MPO) is an enzyme exclusively present in neutrophils granules, which is commonly used as an index of granulocyte infiltration.

The effect of AKP (a compound of formula (I)) as an oral treatment was evaluated in dinitroflurobenzene (DNFB)-induced delayed type hypersensitivity (DTH) in BALB/c mice. A total of 18 female BALB/c mice were randomly assigned to 2 groups, Group 1: DTH treated with the vehicle of AKP (N=9); Group 2: DTH treated with AKP-11 twice daily dose of 3 mg/Kg (N=9). The induction of DTH was conducted as follows: On day 0 and day 1, the shaved mouse abdomen skin was sensitized with 25 μL of 0.5% DNFB in acetone/olive oil (4:1). On day 5, mice were challenged with 20 μL 0.3% DNFB on each side of the right ear. As a control, the mice of Group 1 were dosed with an identical amount of vehicle. Ear thickness was measured with a micrometer before challenge and 24 h after challenge. Ear weight was measured 24 h after challenge. At sacrifice, right ear samples were collected and used for tissue MPO activity.

DNFB induced a significant ear edematogenic response as evidenced by a marked increase in ear thickness and ear pinna weight. Administration of AKP significantly reduced ear thickness and ear weight including the MPO activity.

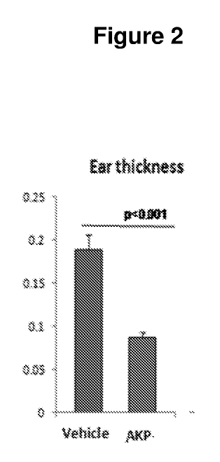

FIG. 2 The in vivo effect of oral AKP administration on dinitrofluorobenzene (DNFB)-induced delayed type hypersensitivity (DTH) in BALB/c mice.

### Example 4

### Topical Efficacy in Inflammatory Skin Disease Model

The formulation of representative compound of formula (I) showed efficacy in phorbol ester mediated inflammation. The local application of Phorbol ester stimulates protein Kinase C (PKC), COX, LOX activity, formation of free radicals including the synthesis of mediators that regulate the production of TNF-α. The whole process mediates inflammation and hyperplasia as observed in inflammatory skin diseases such as psoriasis, eczema and others.

The topical efficacy of AKP (a compound of formula (I)) was determined in the Phorbol ester mediated irritant model of contact dermatitis. The 24 animals (Swiss albino mice) were divided in four groups (G1-4) of 6 each; G1 (control and vehicle treated), G2 (PMA challenged and vehicle treated), G3 (PMA challenged and clobetasol treated) and G4 (PMA challenged and AKP treated). The topical application of AKP (3% formulation; or as a DMSO solution) shown significant inhibition of ear inflammation (>40%; p<0.05). The compound of formula (I) shows synergistic effect with a steroid (betamethasone) to reduce inflammation. The compound of formula (I) or steroid (betamethasone) alone show comparable and significant efficacy and reduced ear swelling (inflammation) by 30% (p>0.05). The combination reduced ear swelling (inflammation) by 43% (p>0.001) as shown in Figure-3 (Topical efficacy of AKP, betamethasone or combination in mice model of Phorbol ester mediated irritant contact dermatitis).

While AKP (the compound of formula (I)) was significantly effective by topical application, it did not alter the count of lymphocytes in the blood.

The corticosteroids are involved in various side effects such as skin atrophy, delayed wound healing, muscle atrophy/myopathy, dermal atrophy, osteoporosis, bone necrosis, glaucoma, pain, interrupt healing (mechanisms involved in the side effects of glucocorticoids, Heike Schacke et al, Pharmacol Therap, 2002, 96, 23-43), lung atrophy (Steroid-induced myopathy and its significance to respiratory disease: a known disease rediscovered, P. N. R. Dekhuijzen, M. Decramer, Eur Respir J 1992, 5, 997-1003).

A S1P receptor mediated mechanism (specifically S1P1) is involved in bone regeneration, (Enhancement of bone regeneration by dual release of a macrophage recruitment agent and platelet-rich plasma from gelatin hydrogels, Yang-Hee Kim et al, Biomaterials, 2014, 35 (1), 214-224) muscle healing (Increased sphingosine-1-phosphate improves muscle regeneration in acutely injured mdx mice, Nicholas leronimakis et al, leronimakis et al. Skeletal Muscle 2013, 3:20), neuronal regeneration (Activation of S1P1 Receptor Regulates PI3K/Akt/FoxO3a Pathway in Response to Oxidative Stress in PC12 Cells, Safarian et al, J Mol Neurosci, 2015, 56, 177), pain alleviation (Bortezomib-induced neuropathic pain is blocked and reversed by blocking the S1P/S1PR1 axis; Stockstill et al, J Pain, 2014, 15(4), S60).

While there is a synergistic effect of AKP (Compound of formula I) with corticosteroid to enhance the efficacy; considering AKP as an S1P1 agonist to counteract the adverse events of corticosteroids (skin atrophy, delayed wound healing, muscle atrophy/myopathy, dermal atrophy, osteoporosis, bone necrosis, glaucoma, pain, interrupt healing) there is an additional advantage in combinations of AKP with corticosteroids. These combinations are useful for dermatological indications (psoriasis, eczema, acne and the like), arthritis related indications (rheumatoid arthritis, gout, osteoarthritis, psoriatic arthritis and the like), lung related indications (asthma, COPD and the like), and eye related indications (Glaucoma, retinopathy and the like), spinal cord injury, and other injuries.

### Example 5

### Clinical Effect of Topical Use in Phase I Clinical Study

The formulation of a representative compound of formula (I) showed efficacy and safety in psoriasis patients. In a clinical study healthy volunteers and psoriasis patients with mild to moderate psoriasis were treated daily for 28 days. There was no serious adverse event and the AKP was not evident in blood in this topical study.

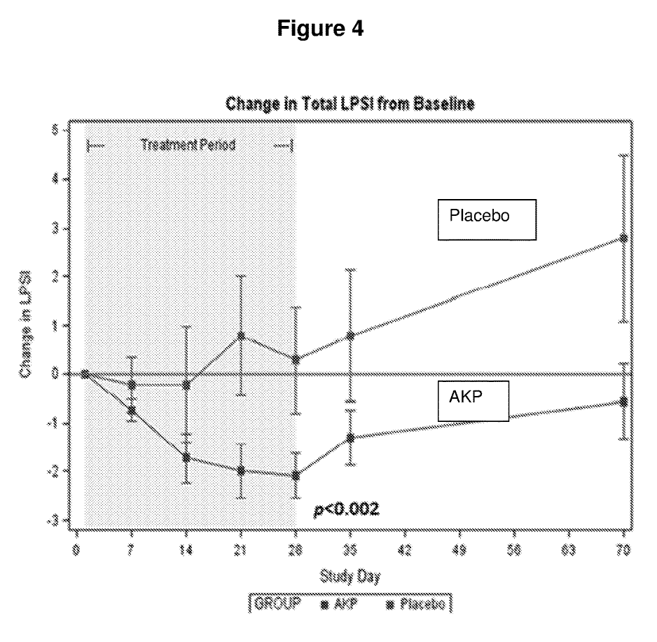

The local psoriasis severity index (LPSI) was measured and recorded significant efficacy (>40%; p=0.0016; Figure-4). FIG. 4 illustrates the clinical effect of AKP.

Compounds of formula (I) have proven S1P receptor activity and other anti-inflammatory effects. Thus compositions comprising compounds of formula (I) and at least one compound selected from one or more of the group consisting of steroids, opioids and non-steroidal anti-inflammatory drugs may be used in a broad range of indications where a broad range of activity is required to address different pathologies with various formulations suitable for such pathologies or indications. These indications may have one or more pathological factors such as an inflammatory site overexpressing the pro-inflammatory cytokines and factors and/or abnormal immune response and/or the blood vessels are abnormal at a disease site and/or VEGF is overexpressed.

### Example 6

### 3% w/w Ointment Composition of S1P1 Agonist of Formula (I), Free Base, for Topical Use.

A mixture of Vaseline (30.8 g) and Gelucire 50/13 pellets (4 g) was melted and stirred at ˜70° C. until homogenous (˜15 min). To it a solution of compound of formula (I), free base, (1.2 g) in anhydrous DMSO (4 ml) was added with vigorous stirring. The mixture was allowed to cool to room temperature to give cloudy ointment (40 g), containing 3% (w/w) of free base of a compound of formula (I).

### Example 7

### 3% w/w Gel Composition HCl Salt of Formula (I), for Topical Use.

A solution of H₂O (4.85 g) and propylene glycol (4.85 g) and cellosize PCG 10 (0.3 g) was prepared. The mixture was allowed to stir overnight at room temperature to give a transparent viscous gel (10 g). This gel (6 g) was mixed with EtOH (4 g) and the resulting mixture was stirred at ˜70° C. for 2 h. To it a hydrochloride salt of a compound of formula (I) (0.45 g), dissolved in anhydrous DMSO (3 g) was added at once and EtOH was added to give a final mass of 15 g. The resulting mixture was stirred for 1 hour at ˜70° C., to give a transparent colourless gel with excellent stability and spread ability.

### Example 8

### 3% w/w Gel Composition of Formula (I), Mesylate Salt, for Topical Use.

When the hydrochloride salt of a compound of formula (I) of Example 7 was substituted for the mesylate salt of a compound of formula (I), an identical process gave the title composition.

### Example 9

### 3% Liquid Composition of Formula (I), Mesylate Salt, for Topical Use.

A mesylate salt of the compound of formula (I) (0.3 g) was dissolved in 50% aqueous DMSO (4 g) and this was diluted to 10 g with EtOH, to give the title formulation as a colourless liquid (10 g).

### Example 10

1% Liquid Composition of Formula (I), HCl Salt, with Polyvinyl Pyrrolidone (PVP) for Topical Use.

A HCl salt of a compound of formula (I) (0.05 g) was dissolved in 80% aqueous EtOH (4.45 ml). To it, polyvinyl PVP (0.5 g) was added and the mixture was stirred until completely homogenous (˜1 h) at room temperature to give a stable colourless solution, which formed a film after application to the skin.

### Example 11

### 0.5% Sterile Aqueous Solution of Formula (I), Mesylate Salt, for Injection/Liquid Oral Formulation/Drops for Eye and Ear Administration.

To a sterile container with a mesylate salt of a compound of formula (I), (0.005 g), sterile isotonic solution was added (1 ml) via syringe and the resulting mixture was stirred at room temperature by shaking until homogenous, which may be used for injection, eye or ear drops or orally.

### Example 12

### Topical Patch Formulation of Formula (I).

A compound of formula (I) and other ingredients including solubility enhancer or permeation enhancer such as and not limited to DMSO, polyvinyl pyrrolidones (PVPs), glycyryl laurates, lauryl lactate, aerosol, eudragit may be dissolved in solvent (ethanol, propanol, isopropanol). An adhesive is added and mixed until homogenous. The homogenous slurry at optimal temperature may be casted onto a release layer (silicone or fluoropolymer coated polyester film and dried.

### Example 13

3% w/w Ointment Composition of S1P1 Agonist of Formula (I), Free Base, in Combination with 1% Nicotinamide and 2% Vitamin E for Topical Use.

A compound of formula (I) as a free base, (0.6 g), nicotinamide (0.2 g), vitamin E (d isomer; 0.4 g), Gelucire 50/13 pellets (2 g), polysorb 20 (0.6 g) in anhydrous DMSO (2 ml) were stirred at ˜55° C. until homogenous (˜30 min). Melted Vaseline was added to make a final weight of 20 g. This was vigorously stirred for 15 min at ˜50° C., cooled to room temperature to give an off white ointment.

### Example 14

### 3% w/w Ointment Composition of S1P1 Agonist of Formula (I), Free Base, and 0.05% w/w of Betamethasone for Topical Use.

A mixture of Vaseline (30.78 g) and Gelucire 50/13 pellets (4 g) was melted and stirred at ˜70° C. until homogenous (˜15 min). To it a solution of compound of formula (I), free base, (1.2 g) and betamethasone (0.02 g) in anhydrous DMSO (4 g) was added with vigorous stirring. The mixture was allowed to cool to room temperature to give cloudy ointment (40 g), containing 3% (w/w) of free base of a compound of formula (I) and 0.05% of betamethasone.

### Example 15

### 2% w/w Gel Composition HCl Salt of Formula (I) and 1% Diclofenac for Topical Use.

A solution of solution of H₂O (4.85 g) and propylene glycol (4.85 g) and cellosize PCG 10 (0.3 g) was prepared. The mixture was allowed to stir overnight at room temperature to give a transparent viscous gel (10 g). This gel (6 g) was mixed with EtOH (3.9 g) and the resulting mixture was stirred at ˜70° C. for 2 h. To it a mixture of hydrochloride salts of a compound of formula (I) (0.3 g) and diclofenac (0.15 g), dissolved in anhydrous DMSO (4.5 g) was added at once and EtOH was added to give a final mass of 15 g. The resulting mixture was stirred for 1 hour at ˜70° C., to give the titled product as a transparent colourless gel with excellent stability and spreadability.

### Example 16

### 2% w/w Gel Composition HCl Salt of Formula (I) and 0.12% Morphine Hydrochloride for Topical Use.

When the hydrochloride salt of diclofenac of Example 15 was substituted for the hydrochloride salt of morphine (18 mg) an identical process gave the title composition.

It is to be understood that while the present disclosure has been described in conjunction with the specific embodiments thereof, the foregoing description is intended to illustrate and not limit the scope of the disclosure. Other aspects, advantages and modifications will be apparent to those skilled in the art to which the disclosure pertains. Therefore, the following examples are put forth so as to provide those skilled in the art with a complete disclosure and description of how to make and use the disclosed compositions, and are not intended to limit the scope of the disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

All documents cited are herein fully incorporated by reference for all jurisdictions in which such incorporation is permitted and to the extent such disclosure is consistent with the description of the present disclosure.

1. A composition comprising at least one S1P receptor modulator and at least one compound selected from one or more of the group consisting of steroids, opioids and non-steroidal anti-inflammatory drugs.

2. A composition according to claim 1, wherein the at least one S1P receptor modulator is a compound of formula (I): 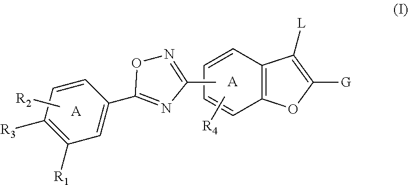
wherein R₁ is selected from hydrogen, deuterium, halogen, CN, CF₃, —COOH, amide, sulphonamide, aryloxy, nitro and an alkyl chain (C₁₋₅), said alkyl chain optionally containing one or more of deuterium, O, S, NR′ (R′ ═H, alkyl, cycloalkyl), halogen, a multiple bond, heterocycle, aryl, cycloalkyl (C₃₋₇) and carbocycle;
wherein R₂ is selected from hydrogen, deuterium, halogen, CN, CF₃, an alkyl chain (C₁₋₄) said alkyl chain optionally containing one or more of deuterium, O, S, NR′ (R′ ═H, alkyl, cycloalkyl), halogen, a multiple bond, heterocycle, aryl or cycloalkyl (C₃₋₇) and carbocycle;
wherein R₃ is selected from hydrogen, deuterium, halogen, an alkyl chain (C₁₋₇) said alkyl chain optionally containing one or more of deuterium, O, S, NR′ (R′ ═H, alkyl, cycloalkyl), halogen, a multiple bond, heterocycle, aryl or cycloalkyl (C₃₋₇) and carbocycle;
wherein R₄ is selected from hydrogen, deuterium, halogen, CN, CF₃, an alkyl chain (C₁₋₄) said alkyl chain optionally containing one or more of deuterium, O, S, NR′ (R′ ═H, alkyl, cycloalkyl), halogen, a multiple bond, heterocycle, aryl and cycloalkyl (C₃₋₇);
wherein A is optional and when present is selected to replace one or more ring carbon atoms by N;
wherein L is selected from hydrogen, deuterium, F, Cl, Br and alkyl (C₁₋₃);
wherein G is a group selected from one of the following: 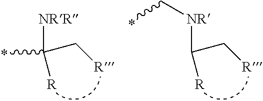
wherein R is selected from H, COOH, alkyl (C₁₋₄) and hydroxy-alkyl (C₁₋₄);
wherein R′ and R″ are independently selected from H and alkyl (C₁₋₄);
wherein R′″ is selected from OH, —OPO₃H₂ and physiologically acceptable salts;
wherein 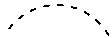
represents an optional bridging group;
the asterisks indicating the attachment of group G within formula (I).

3. A composition according to claim 2, wherein the compound of formula (I) has the formula (II): 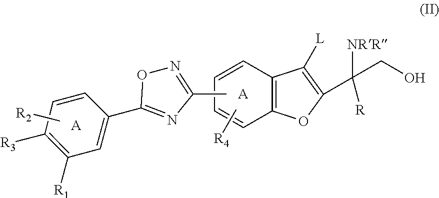
wherein R₁, R₂, R₃, R₄, A, L, R, R′ and R″ are as defined in claim 2.

4. A composition according to claim 2 wherein in the compound of formula (II):
R₁ is selected from F, Cl, Br, CN, CF₃, NO₂, Me, OMe, OEt, OPr, O-iPr, O-isobutyl, O-isopentyl, O-cyclopentyl, O-allyl, O-benzyl and 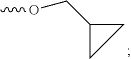
R₂ is selected from H, deuterium, F, Cl, Br, CN, CF₃, NO₂, Me, OMe, OEt, OPr, O-iPr, O-isobutyl, O-isopentyl, O-cyclopentyl, O-allyl, O-benzyl and 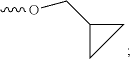
R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and 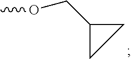
R₄ is selected from H, deuterium, Me and Et;
R is selected from H, Me or —CH₂OH;
R′ is selected from H and Me;
R″ is selected from H and Me;
L is selected from H, deuterium, Me and Cl; and
A is as defined in claim 2.

5. A composition according to claim 2 wherein in the compound of formula (I):
R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and 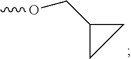
R₂ is H;
R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and 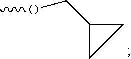
R₄ is selected from H, deuterium, Me and Et;
R is selected from H, Me or —CH₂OH;
R′ is selected from H and Me;
R″ is selected from H and Me;
L is H; and
A is not present.

6. A composition according to claim 3 wherein the compound of formula (I) has the formula (III): 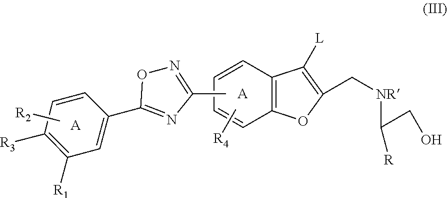
wherein R₁, R₂, R₃, R₄, A, L, R and R′ are as defined in claim 2.

7. A composition according to claim 3, wherein the compound of formula (I) has the formula (III): 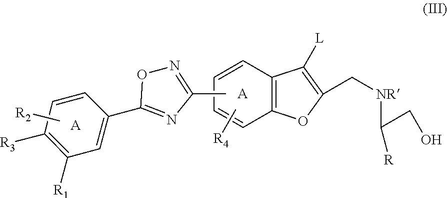
wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and 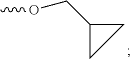
wherein R₂ is selected from H, deuterium, F, Cl, Br, CN, CF₃, Me, OMe, OEt, OPr, O-iPr, O-isobutyl, O-isopentyl, O-cyclopentyl, O-allyl, O-benzyl and 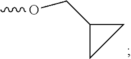
wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and 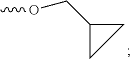
wherein R₄ is selected from H, deuterium, Me and Et;
wherein R is selected from H, Me or —CH₂OH;
wherein R′ is selected from H and Me;
wherein L is selected from H, deuterium, Me and Cl; and
wherein A is as defined in claim 2.

8. A composition according to claim 3, wherein the compound of formula (I) has the formula 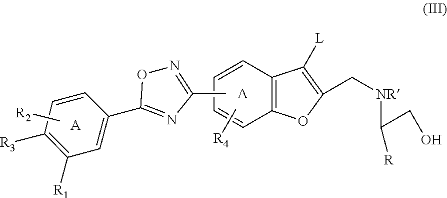
wherein R₁ is selected from F, Cl, Br, CN, CF₃, Me, NO₂, OMe, OEt, OPr, O-iPr, O-isobutyl, O-isopentyl, O-cyclopentyl, O-allyl, O-benzyl and 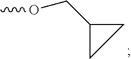
wherein R₂ is H;
wherein R₃ is selected from H, deuterium, Pr, butyl, OMe, OEt, OPr, OiPr, O-isobutyl, O-isopentyl, O-butyl, O-pentyl, O-cyclopentyl, O-allyl, O-benzyl and 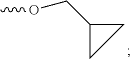
wherein R₄ is selected from H, deuterium, Me and Et;
wherein R is selected from H, Me or —CH₂OH;
wherein R′ is selected from H and Me;
wherein L is H; and
wherein A is not present.

9. A composition according to claim 1, wherein the steroid is a corticosteroid.

10. A composition according to claim 9, wherein the corticosteroid is selected from the group consisting of aclometasone, amcinonide, beclomethasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumethasone, flunisolide, fluocinolone acetonide, fluocinonide, fluocortin, fluocortolone, fluorometholone, fluperolone, fluticasone, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone and ulobetasol, pharmaceutically acceptable salts, esters, solvates, hydrates and derivatives thereof, and mixtures thereof.

11. A composition according to claim 10, wherein the corticosteroid is betamethasone.

12. A composition according to claim 1, wherein the opioid is selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts, solvates, hydrates and derivatives thereof, and mixtures thereof.

13. A composition according to claim 1, wherein the non-steroidal anti-inflammatory drug is selected from the group consisting of aspirin, ibuprofen, naproxen, diclofenac, Cox-2 inhibitors, etodolac, indomethacin, ketoprofen, piroxicam, folmetin, tenoxicam, mecoxicam, meloxicam, mefenamic acid, ibufenac, ketoprofen, pharmaceutically acceptable salts, solvates hydrates and derivatives thereof, and mixtures thereof.

14. A composition according to claim 1, wherein the S1P receptor modulator is present in the composition in an amount of 0.001 wt. % to 25 wt. % and the steroid present in an amount of 0.005 wt. % to 2 wt. %, based on the total weight of the composition.

15. A composition according to claim 11, wherein the S1P receptor modulator is present in the composition in an amount of 0.001 wt. % to 25 wt. % and the betamethasone present in an amount of 0.005 wt. % to 2 wt. %, based on the total weight of the composition.

16. A composition according to claim 15, wherein the S1P receptor modulator is present in the composition in an amount of 1 wt. % to 3 wt. % and betamethasone present in an amount of 0.01 wt. % to 0.05 wt. %, based on the total weight of the composition.

17. A composition according to claim 1, wherein the S1P receptor modulator is present in the composition in an amount of 0.001 wt. % to 25 wt. % and the opioid present in an amount of 0.01 wt. % to 20 wt. %, based on the total weight of the composition.

18. A composition according to claim 1, wherein the S1P receptor modulator is present in the composition in an amount of 0.001 wt. % to 25 wt. % and the non-steroidal anti-inflammatory drug (NSAID) present in an amount of 0.1 wt. % to 20 wt. %, based on the total weight of the composition.

19. A composition according to claim 1, wherein the S1P receptor modulator is present in the composition in an amount of 2 wt. % to 3 wt. % and ibuprofen or diclofenac present in an amount of 1 wt. % to 2 wt. %, based on the total weight of the composition.

20. A composition according to claim 1, wherein the S1P receptor modulator is present in the composition in an amount of 2 wt. % to 3 wt. % and capsaicin present in an amount of 0.01 wt. % to 2.5 wt. %, based on the total weight of the composition.

21. A composition according to claim 1, wherein the S1P receptor modulator is present in the composition in an amount of 2 wt. % to 3 wt. % and lignocaine present in an amount of 0.5 wt. % to 10 wt. %, based on the total weight of the composition.

22. A method of treating or preventing an inflammation mediated disorder, immune mediated disorder or pain by administering to a subject in need thereof an effective amount of a composition according to claim 1.

23. A method according to claim 22, wherein the inflammation mediated disorder or immune mediated disorder is selected from the group consisting of psoriasis, eczema, vitiligo, alopecia, rheumatoid arthritis, osteoarthritis, gout, stroke, haemorrhoid/piles, lung injury, liver injury, acute kidney injury, asthma, chronic obstructive pulmonary disease (COPD), uveitis, retinopathy, nephropathy, macular degeneration, glaucoma, otitis, allergy, sepsis, influenza, rhinitis and pruritus.

24. A method of treating or preventing pain by administering to a subject in need thereof an effective amount of a composition according to claim 1.

25. A method according to claim 24, wherein the pain selected from the group consisting of joint pain, arthritis, gout pain, back pain, muscle pain, neuropathy, neurologic, sports injury pain and wound pain.

26. A method according to claim 22, wherein the composition is administered topically, orally, transdermally, parenterally, intranasally, ocularly or rectally.

27. A method according to claim 22, wherein the composition is in the form of a solid, a patch, a powder, a liquid, a semisolid, an ointment, a gel, a spray, an aerosol, a lotion, a tablet, a capsule, a liquid, a solution, a suspension, an emulsion or a syrup.

28. A method according to claim 22, wherein the composition is a slow release formulation (depot preparation), administered by implantation or injection or device.

29. A method according to claim 22, wherein the composition is administered in combination with other therapeutically active compounds, such as small molecules, biologicals, antivirals, antibacterials, anticancer drugs or other anti-inflammatory agents.
